(19) United States
(12) Patent Application Publication
(10) Pub. No.: US 2008/0138418 A1
(43) Pub. Date: Jun. 12, 2008

(54) Nanoparticles composed of alkyl-cyanoacrylate polymers

(75) Inventors: Yu-Der Lee, Hsinchu City (TW); Chi-Yu Huang, Pingtung County (TW)

Correspondence Address: Joe McKinney Muncy, PO Box 1364, Fairfax, VA, 22038-1364, US

(73) Assignee: TONG SHEN ENTERPRISE CO., LTD.

(21) Appl. No.: 11/634,908

(22) Filed: Dec. 7, 2006

(51) Int. Cl. A61K 9/14; A61K 47/14

(52) U.S. Cl. 424/489; 514/785; 977/788

(57) ABSTRACT

The role of nanoparticle composition as biodegradable carriers for variously therapeutical drugs is disclosed. Nanoparticles are synthesized by anion emulsion polymerization of two alkyl-cyanoacrylate monomers with adjusted content ratio. By modulating the compositions, particle size, hydrophobicity and degradation rate of the copolymers is controlled. Hence, to encapsulate wide range of therapeutical drugs, poly(alkyl cyanoacrylate) nanoparticles with feasible compositions are applied individually. The copolymer nanoparticles produced by n-butyl cyanoactylate (BCA) and 2-octyl cyanoacrylate (OCA), for example, were used therein. The nanoparticles composed of poly[(n-butyl cyanoacrylate)-co-(2-octyl cyanoacrylate)] and poly(2-octyl cyanoacrylate) might be adequate for therapeutical administration.

### FIELD OF THE INVENTION

The present invention relates to compositions of delivery nanoparticles. More particularly, the invention relates to copolymers synthesized by copolymerization of two alkyl cyanoacrylate monomers.

### BACKGROUND OF THE INVENTION

Biodegradable polymeric nanoparticles had been studied and applied as efficient carriers for various drugs, peptides and gene delivery. Many therapeutical agents, particularly those used for oral or intravenous administrations, might undergo degradation gradually and be of low medical efficiency. The appropriate nanoparticles carriers were designed for high loading efficiency and low cytotoxicity. While encapsulated into the nanoparticles, these ingredients were fully protected with the biological stability increased obviously. Recently, various biodegradable polymers, such as poly(lactic acid) (PLA), poly(glycolic acid) (PGA), poly(lactid-co-glycolide) (PLGA) and poly(□-caprolactone) (PCL), had been studied extensively and successfully developed for clinical treatment.

Poly(alkyl-cyanoacrylate) (PACA) was an well-studied therapeutically polymeric compositions for surgical as well as medical applications. PACA was used as alternates or adjuncts to surgical sutures, meshes and staples or other medical devices in wound closure. In addition, PACA was described with capabilities of absorbability and encapsulation for plurality of active agents (U.S. Pat. No. 6,881,421). A wide range of drugs, such as insulin, pilcarpine, vaccines, oligonucleotide and anti-tumor drugs, had been documented to be packaged into the nanoparticles carriers composed of PACA.

Based on clinical studies, only treating with high concentration of anti-cancer drugs might kill cancer cells. However, these drugs possessed serious cytotoxicity. Treating anti-cancer drugs with High concentration not only eliminate cancer but normal cells. Therefore, biodegradable polymeric nanoparticles were used to protect patients. Presently, PACA nanoparticles, especially composed of poly(n-butyl cyanoacrylate) (PBCA), was extensively applied for anti-cancer therapy through intravenous administration. Depend on particle size and surface characteristics of PBCA nanoparticles, encapsulants might deliver to different target organs.

Conventional techniques of nanoparticles synthesis were by emulsion homo-polymerizations. The nanoparticles consisted of PACA were prepared as drug carriers by anion initiated polymerization in stabilizer-containing acidic solution. The significant advantages of the prepared nanoparticles included easy preparation, non-solvent residues remaining and high stability in aqueous medium.

In drug delivery system, release rate of therapeutical drug was controlled by degradation process of nanoparticle carrier. The degradation process of PACA was that alkanol group was released during hydrolysis of the ester chain of alkyl cyanoacrylate monomer. It was indicated in previous studies that several factors, including particle size, the ester chain length, molecular weight of polymer, pH of medium and enzyme used, could influence the degradation rate. The degradation rate of PACA nanoparticles, compared with other biodegradable polymers such as PLA, PLGA, etc., was fast so PACA had less utility as sustained drug release composition. The PLGA copolymer was able to modulate hydrophobicity of nanoparticles by adjusting compositions, but PACA homopolymer was not. Furthermore, the drugs encapsulating and loading efficiency of nanoparticles was related to the compatibility between drugs and nanoparticles. Thus, the therapeutical application of PACA homopolymer might be limited.

### SUMMARY OF THE INVENTION

The present invention provides nanoparticles with better utility for delivering, therapeutical drugs. These nanoparticles are of various properties, such as particle size, hydrophobicity, therapeutical efficiency, cytotoxicity, etc. Because the products of homopolymerization of alkyl cyanoacrylate have narrow application for drug delivery or other application, copolymerizaion of two alkyl-cyanoacrylates was performed. The copolymers composed of the biodegradable nanoparticles were made from two alkyl cyanoacrylate monomers. The PACA copolymers can modulate particle size, hydrophobicity, degradation rate or other characteristics and be provided for various drugs by adjusting content rations. Particularly, PACA copolymers are able to regulate hydrolysis rate to obtain the capability of sustained drug release.

The nanoparticles composition of the present invention is related to copolymers polymerized by alkyl cyanoacrylate monomers with general formula as below:

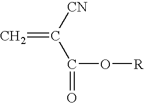

wherein

R=a linear or branched alkyl group (C₂-C₁₀).

The nanoparticles synthesized by anion emulsion copolymerization are formed in spherical shape with small size, less than 100 nm in a narrow distribution. Each composition possesses individual physical and chemical characteristics. Thus, by modulating the contents of compositions, drugs of wide range can be delivered in therapeutical administration. In addition, the POCA nanoparticles, which possess low degradation rate and low cytotoxicity, are used as more feasible carriers for timed-release drug.

### BRIEF DESCRIPTION OF THE DRAWINGS

FIG. 1 shows a Scanning Electro Micrograph (SEM) picture of POCA nanoparticles.

FIG. 2 shows degradation of poly(BCA-co-OCA) nanoparticles in phosphate buffer solution (PBS; pH7.4) at 37° C.: (a) butanol and 2-octanol yield (mean±S.D., n=3) presented as a molar percent of the total BCA and OCA units initially present in the nanoparticles; (b) butanol concentration (mean±S.D., n=3); (c) 2-octanol concentration (mean±S.D., n=3).

FIG. 3 shows in vitro cytotoxicity (mean±S.D., n=3) for poly(BCA-co-OCA) nanoparticles with varying BCA/OCA (w/w) composition at the concentration of 10 □g/ml.

### DETAILED DESCRIPTION OF THE PREFERRED EMBODIMENTS

Copolymer compositions of the present invention produced therefrom are used for delivering therapeutical drugs. More particularly, these nanoparticles can control releasing rate of the encapsulated active agents by their own appropriate properties through adjusting their content ratio of copolymerization.

The nanoparticle copolymers of the present invention are comprised two alkyl cyanoacrylate monomers with general formula (I) as below:

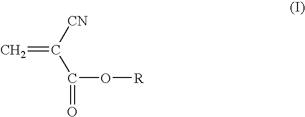

wherein

R=a linear or branched alkyl group (C₂-C₁₀).

The following examples of the two monomers for the synthesis of cyanoacrylate polymers are illustrative, but not limiting the scope of the present invention. Reasonable variations, such as those occur to reasonable artisan, can be made herein without departing from the scope of the present invention.

Two monomers produced cyanoacrylate copolymers of general formula (II) and (III) as below:

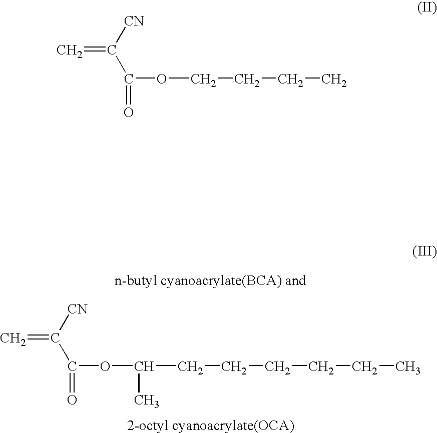

The copolymers polymerized by BCA and OCA are poly[(n-butyl cyanoacrylate)-co-(2-octyl cyanoacrylate)] (poly(BCA-co-OCA)). To prepare the poly(BCA-co-OCA) nanoparticles, monomers were mixed well and dispersed into polymerization media respectively. Anion emulsion polymerization was used to prepare the copolymers, and the detailed methods were described as below:

The nanoparticles of PBCA, POCA and poly(BCA-co-OCA) with compositions of BCA/OCA ratio: 100/0, 75/25, 50/50, 25/75 and 0/100% (wt. %/wt. %) were prepared individually by emulsion polymerization technique.

About 0.5 g of monomer or monomers mixture of n-butyl cyanoacrylate and 2-octyl cyanoacrylate was added drop by drop to a 50 ml aqueous solution of 0.01 N hydrochloric acid containing 0.3% (w/v) of Pluronic F 127 [poly(ethylene oxide)-poly(propylene oxide)-poly(ethylene oxide) triblock copolymer; MW 12,6000 Da]under stirred with a magnetic stirrer. The mixtures were stirred for 18 hr in a 100 ml double-walled round-bottomed reactor surrounded by a thermostatic water bath set at 25° C. Then a solution of IN sodium hydroxide solution was added to neutralize the suspension, followed stirring for another 30 min. The nanoparticles formed were separated by ultracentrifugation at 60,000×g for 60 min (CP 100 MX, Hitachi, Japan) and redispersed in water and lyophilized.

Freeze-dried nanoparticles were dissolved in acetone (20 mg/ml). Then water was added in steadily until the color of the solution changed to milky white. The copolymer was collected by centrifugation at 10,000×g for 10 min. the purified copolymer was obtained by repeating the procedure three times and collected.

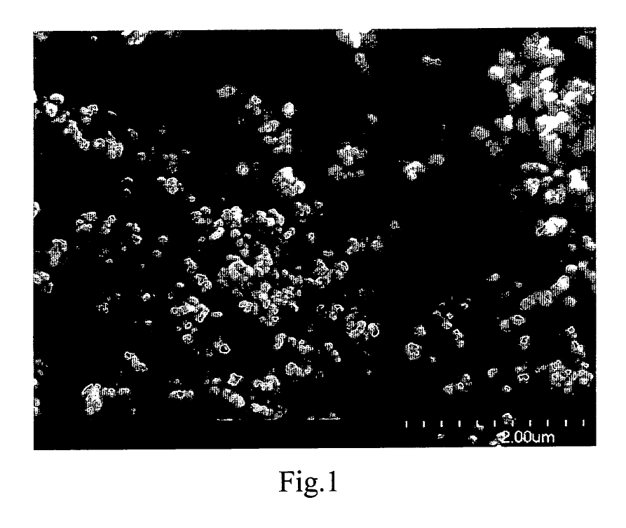

The collected polymerization products were observed by scanning electron microscopy (SEM) analysis. SEM was conducted using a FESEM; Hitachi S-4700. Samples of collected products were placed on a 400 mesh carbon coated with cooper grid. After drying, the samples were observed at 15 kV. The prepared nanoparticles were spherical shape with narrow distribution and did not show any aggregation (FIG. 1).

Then, these products were processed several characteristics, including particle size, polydispersity index (PI) and zeta potential.

The particle size and the zeta potential of nanoparticles were measured by photon correlation spectroscopy (PCS; Zetasizer 3000, Malven Instruments, Malvern, UK) at 25° C. The scattered light of wavelength 633 nm was detected at an angle of 90°. The nanoparticles dispersion was diluted by water to an adequate concentration to facilitate measurement. The mean size of hydrodynamic particle was represented as the value of z-average size. The width of the size distribution was indicated by polydispersity index. The results are presented in table 1 below.

TABLE 1

| Feed composition | | | | |
|---|---|---|---|---|
| (wt. %) | | Particle | Polydispersity | Zeta potential |
| BCA | OCA | size (nm) | index | (mV) |
| 100 | 0 | 74.3 ± 1.0 | 0.121 | −21.6 ± 0.6 |
| 75 | 25 | 84.3 ± 3.1 | 0.104 | −25.0 ± 0.1 |
| 50 | 50 | 89.6 ± 3.4 | 0.103 | −24.9 ± 2.1 |
| 25 | 75 | 94.5 ± 0.6 | 0.082 | −25.2 ± 1.3 |
| 0 | 100 | 98.1 ± 0.3 | 0.076 | −27.7 ± 0.5 |

Particle size, polydispersity index and zeta potential were influenced by the ration of BCA/OCA.

While OCA weight content increased, the particle size of nanoparticles increased cooresponding, but the values of polydispersity index decreased. The OCA molecules containing octyl groups were more hydrophobic and nonpolar than BCA molecules. High polarity of the polymer tended to stabilize the surface energy of particles. Therefore, the nanoparticles size increased with increasing the content of OCA. In previous studies, the degree of second nucleation was low for hydrophobic monomer in emulsion polymerization. So the presence of OCA decreased the polydispersity index of nanoparticles. In addition, zeta potential is the electrostatic potential of particle surface generated by ions accumulation. The negative charge increased slightly as the content of OCA increased.

The degradation rate of nanoparticles was analyzed by the concentration of alkanol hydrolyzed from poly(alkyl cyanoacrylate).

Lyophilized nanoparticles were redispersed in pH 7.4 PBS at a concentration of 2 mg/ml and placed in a shaking incubator (60 r.p.m.) at 37° C. At different time intervals, the nanoparticles were separated from dispersion media by ultracentrifugation (CP100 MX, Hitachi, Japan) at 60,000×g for 60 min. A 5 ml sample of supernatant was withdrawn from the dispersion, and mixed with 1□1 n-hexanol (internal standard). This solution was extracted by 2.5 ml of diethyl ether which was then injected into gas chromatography (HP5890, USA) coupled with a mass spectrometer (HP 5972, USA). The GC column used was a DB-5MS (39 m×0.25 mm i.d. and a film thickness 0.25 □m) capillary column. Carrier gas was helium at a flow rate of 1.5 ml/min. The oven temperature was ramped from 40 to 300° C. and held for 3 min. The temperature of the injector and detector were set as 220 and 280° C., respectively.

The concentrations of alkanols were determined from the calibration curves. Standard solutions of 0.003, 0.01, 0.05, 0.1 and 0.2 mg/ml of alkanols were prepared separately for calibration. The peak area ratios (alkanol/n-hexanol) were analyzed from chromatographic patterns against the concentration of the respective calibration standards.

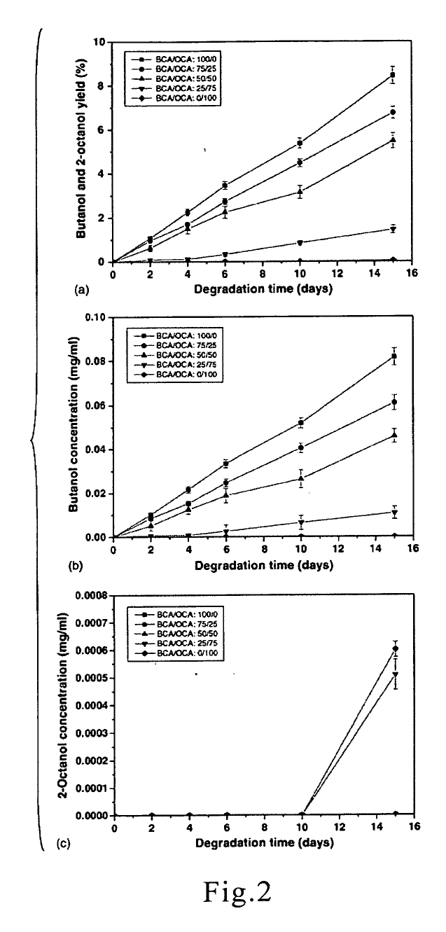

The degradation rates of poly(BCA-co-OCA) nanoparticles were presented in FIG. 2. The value of degradation rate of POCA was much lower than PBCA only (FIG. 2a). As OCA content raised, in addition, the concentrations of the yield alknols significantly decreased (FIG. 2b and FIG. 2c). Therefore, the hydrolytic rate of poly(BCA-co-OCA) nanoparticles could be modulated to be of wide range by adjusting the content of OCA in the copolymer. The presence of OCA only, particularly, possesses the lowest alkanols concentration and degradation rate. While active agents encapsulated into OCA nanoparticles, these encapsulants might be release sustainedly.

To evaluate the in vitro cytotoxicity of the poly(BCA-co-OCA) nanoparticles was used the 3-(4,5-dimethylthiazol-2-yl)-2,5-diphenyltetrazolium bromide (MTT) assay for viability of the human foreskinon fibroblastic HS 68 cells viability for 3 days. Sterile nanoparticles suspension were diluted by Dulbecco's modified Eagle medium (DMEM) supplemented with 10% fetal bovine serum (FBS) and 1% penicillin/streptomycin (10⁴ IU/ml) to the concentration of 10 □g/ml. Fibroblastic cells were seeded at a density of 5×10⁴ cells per well in a 24-well plate, and grew for 24 hr. Thereafter, the cells were washed with PBS (pH 7.4) and incubated with diluted nanoparticles suspension for 3 days at 37° C. and 5% (v/v) CO₂. After incubation, the upper medium was carefully removed and cells were washed twice with PBS. Then, 1 ml MTT solution (0.5 mg/ml) was added to each well and incubated for another 4 hr. The intracellular blue formazon salt metabolizing the MTT by live cells was dissolved by adding 1 ml dimethylsufoxide. The absorbance values were measured by a multiwell microplate reader (SUNRISE TS, TECAN) at a wavelength of 570 nm. The relative cell viability (%) in comparison with control well containing cell culture medium without nanopartiles was calculated by the following equation:

$$relative cell viability ( \% ) = \frac{{[ absorbance ]}_{test}}{{[ absorbance ]}_{control}} \times 100$$

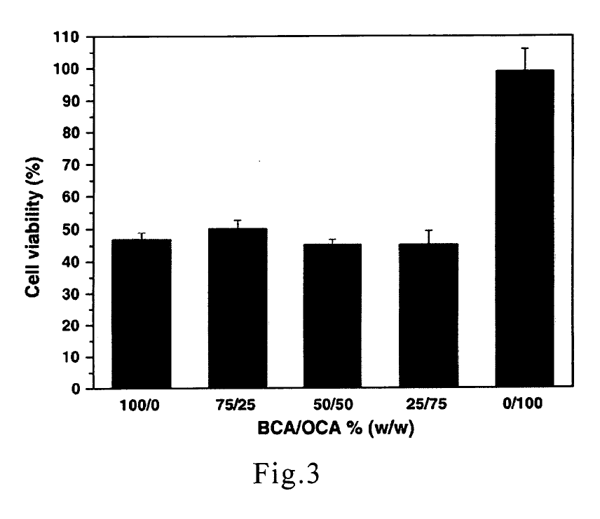

The results of the in vitro cytotoxicities of PBCA, POCA and poly(BCA-co-OCA) nanoparticles were presented in FIG. 3.

Comparing with the PBCA, POCA nanoparticles did not affect the viability of human foreskin fibroblasts, indicating their low cytotoxicity. However, except POCA nanopartilces only, there was little difference between the toxicities of the copolymer nanoparticles. These results indicated that the cytotoxicity of POCA nanoparticles was quite low compared to PBCA nanoparticles.

In one embodiment of the present invention, POCA nanoparticles might be more feasible to apply in therapeutical administration. Comparing with other copolymers, POCA nanoparticles obtained the lowest degradation rate and rare cytotoxicity during hydrolysis. Concerning with medical safety, therefore, POCA was better used in production of therapeutically delivery nanoparticles.

Furthermore, the contents of monomers polymerized into poly(BCA-co-OCA) were not limited only BCA/OCA ratio: 75/25, 50/50 and 25/75% (w/w), but including 0/100 above to 100/0 below %(w/w).

While the invention has been described by way of examples and in terms of the preferred embodiments, it is to be understood that the invention is not limited to the disclosed embodiments. On the contrary, it is intended to cover various modifications as would be apparent to those skilled in the art. Therefore, the scope of the appended claims should be accorded the broadest interpretation so as to encompass all such modifications.

What is claimed is:

1. A copolymer composition for carrying biological or threapeutical agents comprising two alkyl cyanoacrylate monomers with the formula as below: 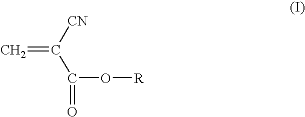
wherein
R=a linear or branched alkyl group (C₂-C₁₀).

2. The copolymer composition of claim 1, wherein the content of each alkyl cyanoacrylate monomer in nanoparticles is more than 0% (w/w) and less than 100% (w/w).

3. The copolymer composition of claim 1, wherein the nanoparticle sizes are less than 100 nm with narrow size distribution.

4. The copolymer compositions of claim 1, wherein the biological or therapeutical carriers control the release rates of encapsulated drugs.

5. A homopolymer composition for carrying biological or threapeutical agents comprising 2-octyl cyanoacrylate monomer.

6. The homopolymer compositions of claim 5, wherein the nanoparticle sizes are less than 100 nm with narrow size distribution.

7. The homopolymer compositions of claim 5, wherein the biological or therapeutical carriers have low cytotoxicity and sustainedly release encapsulated drugs.
